SEQUENCE LISTING (6 sequences)

SEQ ID NO: 1 - DNA, 13 bp, synthetic construct
  misc_feature 1..13: note = Synthetic Polynucleotide
  source 1..13: mol_type = other DNA
catagtacctagg

SEQ ID NO: 2 - DNA, 13 bp, synthetic construct
  misc_feature 1..13: note = Synthetic Polynucleotide
  source 1..13: mol_type = other DNA
cataggacctagg

SEQ ID NO: 3 - DNA, 13 bp, synthetic construct
  misc_feature 1..13: note = Synthetic Polynucleotide
  source 1..13: mol_type = other DNA
aagaacaagacag

SEQ ID NO: 4 - DNA, 33 bp, synthetic construct
  misc_feature 1..33: note = Synthetic Polynucleotide
  source 1..33: mol_type = other DNA
gctgatgtgccgcctcacttcggtggtgaggtg

SEQ ID NO: 5 - DNA, 32 bp, synthetic construct
  misc_feature 1..32: note = Synthetic Polynucleotide
  source 1..32: mol_type = other DNA
ctgatgtgccgcctcacttcggtggtaccgta

SEQ ID NO: 6 - DNA, 32 bp, synthetic construct
  misc_feature 1..32: note = Synthetic Polynucleotide
  source 1..32: mol_type = other DNA
acgaggtgccgcctcacttcggtggtgaggtg

(19) United States
(12) Patent Application Publication
(10) Pub. No.: US 2025/0299778 A1
(43) Pub. Date: Sep. 25, 2025

(54) SYSTEMS AND METHODS FOR ALIGNING SEQUENCES TO GRAPH REFERENCE CONSTRUCTS

(71) Applicant: Seven Bridges Genomics UK, Ltd., London (GB)

(72) Inventors: Björn Pollex, London (GB); Iosif Daniel Spulber, London (GB); Amit Jain, London (GB)

(73) Assignee: Seven Bridges Genomics UK, Ltd., London (GB)

(21) Appl. No.: 19/071,665

(22) Filed: Mar. 5, 2025

Related U.S. Application Data
(63) Continuation of application No. 15/452,963, filed on Mar. 8, 2017, now abandoned.

(51) Int. Cl. G16B 30/10; G16B 30/00; G16B 45/00

(52) U.S. Cl. CPC G16B 30/10; G16B 30/00; G16B 45/00

(57) ABSTRACT

Techniques for aligning a biological sequence to a graph reference construct. The graph reference construct includes first, second, and third nodes. The techniques may include: accessing first state data indicating an extent to which each of multiple subsequences of the biological sequence matches the construct when aligned so as to end at a last position of a sequence represented by the first node; accessing second state data indicating an extent to which each of the multiple subsequences matches the construct when aligned so as to end at a last position of a sequence represented by the second node; and generating third state data using the first state data and the second state data, the third state data indicating an extent to which each of the multiple subsequences matches the construct when aligned so as to end at a first position of a sequence represented by the third node.

### FIELD

Aspects of the technology described herein relate to systems and methods for aligning biological sequences to graph reference constructs.

### BACKGROUND

Advances in sequencing technology, including the development of next generation sequencing methods, have made sequencing an important tool used both in research and in medicine. Some applications of sequencing technology include aligning the sequence reads obtained by sequencing techniques against a reference sequence construct, and identifying the differences, sometimes termed “variants,” between the sequence reads and the reference sequence construct. In turn, the identified differences may be used for diagnostic, therapeutic, research, and/or other purposes.

There are different types of reference sequence constructs to which sequence reads may be aligned. For example, sequence reads may be aligned against a linear reference sequence construct such as, for example, the hg19 or hg38 human reference genomes. As another example, sequence reads may be aligned against a reference sequence construct that accounts for one or more known variants at one or more respective locations. One example of such a reference sequence construct is a graph-based reference sequence construct (sometimes referred to herein as a “graph reference construct” or a “graph reference”). A graph reference may represent a graph (e.g., a directed acyclic graph) through which there may be multiple paths, each of which may represent one or multiple known variants.

An illustrative example of a graph reference construct is shown in FIG. 1, which depicts a graph reference construct 100. Graph reference 100 includes a directed acyclic graph comprising nodes 102, 104, 106, 108, 110, 112, 114, and 116, and directed edges connecting these nodes. Each of the nodes in graph reference construct 100 represents a respective sequence. In this example, node 102 represents the sequence “CATAG”, node 104 represents the sequence “T”, node 106, represents the sequence “G”, node 108 represents the sequence “ACCTAGG”, node 110 represents the sequence “GG”, node 112 represents the sequence “TCTTGG”, node 114 represents the sequence “AG”, and node 116 represents the sequence “CTAGTC”. As may be appreciated from the example of FIG. 1, a node in a graph reference may represent a sequence consisting of a single nucleotide (e.g., node 104 represents the single nucleotide sequence “T”) or multiple nucleotides (e.g., node 112 represents the multi-nucleotide sequence “TCTGG”).

The directed acyclic graph of a graph reference construct may represent genetic variation in a population. Genetic variation in a sequence may be represented using different paths through alternate nodes of the graph. For example, the graph reference construct 100 shows that, after the sequence “CATAG” that is represented by node 102, either a “T” or a “G” may follow, as indicated by alternate paths through node 104 (representing “T”) or node 106 (representing “G”), before the subsequent sequence “ACCTAGG” that is represented by node 108. As such, the nodes 102, 104, 106, and 108 of graph reference 100 represent the genetic sequence “CATAGTACCTAGG” (SEQ ID NO: 1) and the genetic sequence “CATAGGACCTAGG” (SEQ ID NO: 2). The first sequence is represented through the path defined by nodes 102, 104 and 108, whereas the second sequence is represented through the path defined by nodes 102, 106, and 108. As can be appreciated through this example, different paths through a graph reference construct represent genetic variation and the associated sequences that embody such variation. Aspects of graph reference constructs are further discussed in U.S. Patent Publication No. 2015-0057946, entitled “METHODS AND SYSTEMS FOR ALIGNING SEQUENCES,” published on Feb. 26, 2015, which is incorporated by reference herein in its entirety.

### SUMMARY

Some embodiments are directed to a system comprising: at least one computer hardware processor; and at least one non-transitory computer-readable storage medium storing processor-executable instructions that, when executed by the at least one computer hardware processor, cause the at least one computer hardware processor to perform a method. The method comprises: accessing a biological sequence; accessing a graph reference construct representing a graph through which there are multiple paths including a first path and a second path, the graph comprising a plurality of nodes including first, second, and third nodes, the first node preceding the third node along the first path, and the second node preceding the third node along the second path; and aligning the biological sequence to the graph reference construct. The aligning comprises: accessing first state data indicating an extent to which each of multiple subsequences of the biological sequence matches the graph reference construct when aligned to the graph reference construct so as to end at a last position of a sequence represented by the first node; accessing second state data indicating an extent to which each of the multiple subsequences of the biological sequence matches the graph reference construct when aligned to the graph reference construct so as to end at a last position of a sequence represented by the second node; and generating third state data using the first state data and the second state data, the third state data indicating an extent to which each of the multiple subsequences of the biological sequence matches the graph reference construct when aligned to the graph reference construct so as to end at a first position of a sequence represented by the third node; and storing the third state data.

Some embodiments are directed to a method, comprising: using at least one computer hardware processor to perform: accessing a biological sequence; accessing a graph reference construct representing a graph through which there are multiple paths including a first path and a second path, the graph comprising a plurality of nodes including first, second, and third nodes, the first node preceding the third node along the first path, and the second node preceding the third node along the second path; and aligning the biological sequence to the graph reference construct. The aligning comprises: accessing first state data indicating an extent to which each of multiple subsequences of the biological sequence matches the graph reference construct when aligned to the graph reference construct so as to end at a last position of a sequence represented by the first node; accessing second state data indicating an extent to which each of the multiple subsequences of the biological sequence matches the graph reference construct when aligned to the graph reference construct so as to end at a last position of a sequence represented by the second node; generating third state data using the first state data and the second state data, the third state data indicating an extent to which each of the multiple subsequences of the biological sequence matches the graph reference construct when aligned to the graph reference construct so as to end at a first position of a sequence represented by the third node; and storing the third state data.

Some embodiments are directed to at least one non-transitory computer-readable storage medium storing processor-executable instructions that, when executed by at least one computer hardware processor, cause the at least one computer hardware processor to perform: accessing a biological sequence; accessing a graph reference construct representing a graph through which there are multiple paths including a first path and a second path, the graph comprising a plurality of nodes including first, second, and third nodes, the first node preceding the third node along the first path, and the second node preceding the third node along the second path; and aligning the biological sequence to the graph reference construct, the aligning comprising: accessing first state data indicating an extent to which each of multiple subsequences of the biological sequence matches the graph reference construct when aligned to the graph reference construct so as to end at the first node; accessing second state data indicating an extent to which each of the multiple subsequences of the biological sequence matches the graph reference construct when aligned to the graph reference construct so as to end at the second node; generating third state data using the first state data and the second state data, the third state data indicating an extent to which each of the multiple subsequences of the biological sequence matches the graph reference construct when aligned to the graph reference construct so as to end at the third node; and storing the third state data.

### BRIEF DESCRIPTION OF DRAWINGS

Various aspects and embodiments will be described with reference to the following figures. The figures are not necessarily drawn to scale.

FIG. 1 is an illustrative diagram of a graph-based reference sequence construct.

FIG. 2A is a diagram of an illustrative graph reference construct, in accordance with some embodiments of the technology described herein.

FIG. 2B is a diagram illustrating state data used to align the illustrative sequence “AACCGA” to the example graph reference construct of FIG. 2A, in accordance with some embodiments of the technology described herein.

FIG. 3 is a diagram illustrating a bit-parallel automaton (BPA) alignment technique, in accordance with some embodiments of the technology described herein. FIG. 3 includes SEQ ID NO: 3.

FIG. 4A is a diagram of another illustrative graph reference construct, in accordance with some embodiments of the technology described herein.

FIG. 4B is a diagram illustrating state data used to align the illustrative sequence “AACAG” in accordance with some embodiments of the technology described herein.

FIG. 5 is a flowchart of an illustrative process for aligning a biological sequence to a graph reference construct, in accordance with some embodiments of the technology described herein.

FIG. 6 is a block diagram of an illustrative computer system that may be used in implementing some embodiments of the technology described herein.

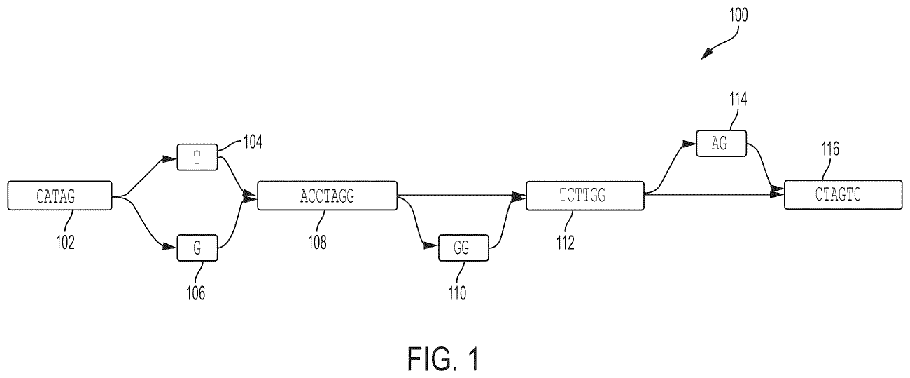

### DETAILED DESCRIPTION

Aligning biological sequence reads against a graph reference, which accounts for known genetic variations among people, aids accurate placement of sequence reads and facilitates identification of variants based on results of the alignment. However, the inventors have recognized that conventional techniques for aligning sequence reads to graph references may be improved upon because they are computationally expensive. Although, some computational shortcuts and approximations may be used to speed up the computation in limited circumstances, such approaches are undesirable because they may lead to inaccurate results.

For example, some conventional techniques for aligning a biological sequence to a graph reference involve using a linear alignment algorithm to compute a linear alignment between the biological sequence and each path through the graph reference. The computational complexity of such a strategy depends on the number of paths through the graph reference. However, the number of paths through a graph reference is exponential in the number of variants represented by the graph reference and a graph reference typically represents a very large number of variants. As a consequence, computing a linear alignment between a biological sequence and each path through the graph reference is computationally infeasible for all but the smallest (and least useful) graph references. For example, the 1000 Genomes Project performed whole-genome sequencing of a geographically diverse set of 2,504 individuals, yielding a broad spectrum of genetic variation including over 88 million known variants. Incorporating all of these variants into a single graph reference yields regions of the graph that include a very large number of paths (reflecting significant variation in corresponding regions of the human genome). Aligning a biological sequence to such a graph reference (or portions thereof), by performing a linear alignment against each of the paths through the graph reference is computationally infeasible.

Accordingly, the inventors have developed a new class of techniques for aligning biological sequences against graph references, which do not involve aligning biological sequences against each individual path through a graph reference. Rather the new class of techniques involves performing alignment by traversing the graph underlying the graph reference (e.g., using breadth-first search) and using a linear alignment algorithm suitably augmented in order to handle branching and merging in the graph. As described in more detail below, such augmentation may be achieved by storing state information for each node in the graph and, in some embodiments, may involve storing state information for each position of the sequence represented by a node in the graph. The inventors recognized that any of numerous types of linear alignment algorithms may be augmented in this manner and be used for efficient alignment biological sequences to graph references. Non-limiting examples of such linear alignment algorithms include the bit parallel automaton (BPA) alignment algorithm and the Smith Waterman alignment algorithm.

Notably, the computational complexity of the alignment techniques developed by the inventors is linear in the number nodes in the graph underlying the graph reference, whereas the computational complexity of conventional techniques that examine each path through the graph depends is exponential in the number of nodes in the graph. (When one or more of the nodes of a graph reference represent multi-nucleotide sequences, the computational complexity of the alignment techniques developed by the inventors is linear in the number of sequence positions represented by the nodes in the graph.) The techniques developed by the inventors for aligning sequence reads to a graph reference reduce the overall computational complexity of performing such an alignment and lead not only to a decrease in the time required to perform the alignment, but also to an increase in its accuracy because the computational complexity of conventional techniques required not examining dense graph regions at all, which leads to errors, and using the techniques described herein allows these regions to be examined leading to improved accuracy.

Some embodiments described herein address all of the above-described issues that the inventors have recognized with conventional techniques for aligning biological sequences to a graph reference. However, not every embodiment described herein addresses every one of these issues, and some embodiments may not address any of them. As such, it should be appreciated that embodiments of the technology described herein are not limited to addressing all or any of the above-discussed issues of conventional techniques for aligning biological sequences to a graph reference. It should be appreciated that the various aspects and embodiments described herein be used individually, all together, or in any combination of two or more, as the technology described herein is not limited in this respect.

Accordingly, in some embodiments, a biological sequence may be aligned to a graph reference by using a linear alignment algorithm modified to handle branches and merges in the graph reference. The modification may involve augmenting the linear alignment algorithm to generate and keep track of additional information that allows the aligner to take graph branches and merges into account. In some embodiments, generating the additional information involves generating state data for each of one or more positions of each sequence represented by a node of the graph reference construct. In such embodiments, aligning a biological sequence to a graph reference construct may comprise iteratively traversing nodes of the graph underlying the graph reference (e.g., using breadth-first search) and generating state data for one or more positions of each sequence represented by a node of the graph reference construct. For example, aligning a biological sequence to the graph reference construct 100 may involve iteratively generating state data for each nucleotide in the sequence represented by node 102, 104, 106, 108, 110, 112, 114, and 116. As discussed herein, the state data for a particular position of a sequence represented by a node in the graph may be generated using the biological sequence being aligned, the sequences represented by node of the reference construct, and the state data computed for one or more preceding positions in the graph. In turn, the generated state data may be used to identify the best alignment(s) of the biological sequence to the graph reference, and to resume alignment without having to re-compute previous partial alignments.

In some embodiments, state data for a nucleotide at a particular position in a sequence represented by a node in a graph reference may be generated using state data obtained for one or more preceding positions in the graph. As one example, state data for a nucleotide at a position other than the first position in a sequence represented by a node may be generated using state data generated for the nucleotide at a preceding position in the same sequence. As a specific non-limiting example, state data for the nucleotide “C” at the second position of the sequence “ACCTAGG” represented by node 108 may be generated using state data generated for the nucleotide “A” at the first position of the same sequence. As another specific non-limiting example, state data for the nucleotide “G” at the last position of the sequence “TCTGG” represented by node 112 may be generated using state data generated for the nucleotide “G” at the second-to-last position of the same sequence.

As another example, state data for a nucleotide at a first position in a sequence represented by a particular node may be generated using state data generated for the nucleotide(s) at the last position of the sequence(s) represented by the node(s) preceding the particular node in the graph. As one specific non-limiting example, state data for the nucleotide “T” at the first position of the sequence represented by node 104 may be generated using state data for the nucleotide “G” at the last position of the sequence “CATAG” represented by node 102. As another specific non-limiting example, state data for the nucleotide “A” at the first position of the sequence “ACCTAG” represented by node 108 may be generating using: (1) state data for the nucleotide “T” at the last position of the sequence represented by node 104; and (2) state data for the nucleotide “G” at the last position of the sequence represented by node 106.

In some embodiments, when two paths through the graph reference merge at a particular node (e.g., node 108) using the state data from the two nodes preceding the particular node (e.g., nodes 104 and 106) to generate state data for a first position of the sequence represented by the particular node involves: (1) accessing state data for the nucleotide at the last position of the sequence represented by the first node (e.g., node 104) preceding the particular node (e.g., node 108), which may be termed “first state data”; (2) accessing state data for the nucleotide at the last position of the sequence represented by the second node (e.g., node 106) preceding the particular node (e.g., node 108), which may be termed “second state data”; and (3) generating state data for the first position of the sequence represented by the particular node (e.g., node 108) using the first state data and the second state data.

In some embodiments, the third step of generating the state data for the first position of the sequence represented by the particular node may include: (1) merging the first state data and the second state data to obtain merged state data; and (2) updating the merged state data to account for the identity of the nucleotide at the first position of the sequence represented by the particular node.

In some embodiments, state data for a nucleotide at a particular position in a sequence represented by a node in a graph reference may indicate an extent to which each of one of multiple subsequences of the biological sequence matches the graph reference construct when aligned to the graph reference construct so as to end at the particular position. Such matches and corresponding state data may be termed “partial alignments,” as each represents a partial match of a subsequence to the graph reference construct. State data may indicate the extent of a match between two sequences when aligned in a given way to one another (e.g., the extent of a match between a prefix of a biological sequence and the sequence represented by a node of a graph reference construct when the prefix is aligned to the graph reference so as to end at a particular position of the sequence represented by the node) in any suitable way, two illustrative non-limiting examples of which are described below.

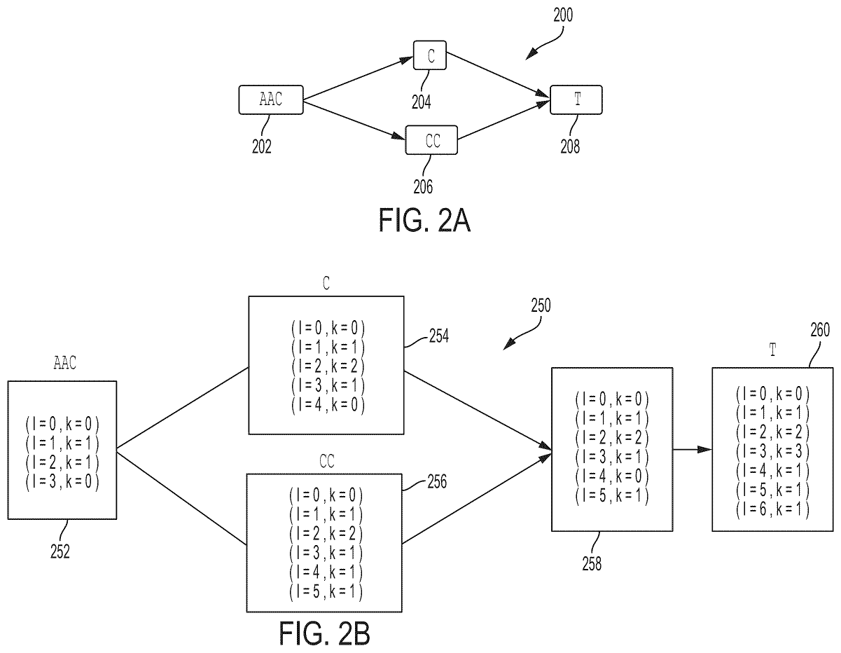

As a first example, state data may indicate the extent of a match between two sequences by providing an indication as to whether there is an exact match between the two sequences. For example, the state data may include a “0” indicating that there is no exact match or a “1” indicating that there is an exact match, or vice versa, or in any other suitable way (e.g., without using a binary value). As one specific non-limiting example, consider aligning the sequence “AACCGA” to the graph reference 200 shown in FIG. 2A. Graph reference 200 includes node 202 (representing the sequence “AAC”), node 204 (representing “C”), node 206 (representing “CC”), and node 208 (representing “T”). Aligning “AACCGA” to the graph reference 200 may involve computing state data for the nucleotide “C” at the last position of the sequence “AAC” represented by node 202. In this example, the state data may provide an indication, for each of multiple prefixes of the biological sequence “AACCGA,” of whether the prefix matches the sequence “AAC” represented by node 202 when aligned so as to end at the last position of “AAC.” For example, as illustrated in Table 1 below, the state data may indicate that there is no match when the subsequences “A” and “AA” are aligned so as to end on the last position of “AAC,” but that there is a match, when the subsequence “AAC” is so aligned.

TABLE 1
State Data (2^{nd} column) indicates whether there is an exact match
between various prefixes of “AACCGA” (i.e., “A”, “AA”, and
“AAC”) and the sequence “AAC” represented by node 202 of graph
reference construct 200 of FIG. 2, when the prefix is aligned to the
graph reference so as to end at the last position of “AAC.” A “1”
indicates an exact match and a “0” indicates no exact match.

| Partial Alignment Ending at “C,” which | |
|---|---|
| is the Last Position of “AAC” | State Data |
| AAC | 0 (No Exact Match) |
| A | |
| AAC | 0 (No Exact Match) |
| AA | |
| AAC | 1 (Exact Match) |
| AAC | |

As may be appreciated from this example, the state data for a position of a sequence represented by a node in the graph reference may be binary data. In the binary data, a 1 may indicate an exact match for a partial alignment and a 0 may indicate that there is not an exact match for the specific partial alignment (or vice versa). The binary data may be stored in any suitable format, as aspects of the technology described herein are not limited in this respect.

As a second example, state data may indicate the extent of a match between two sequences by providing an indication as to how many errors there are between the two sequences when in a given alignment relative to one another. For example, state data for position P at node N of a graph reference may indicate how many errors there are between a prefix of a biological sequence and the sequence represented by the graph reference that starts at the left-most node and ends at position P of node N, when the prefix is aligned so as to end at position P of node N. As a specific non-limiting example, the state data may provide an indication, for each of multiple prefixes of the biological sequence “AACCGA,” of how many errors there are between the prefix and the sequence “AACC” represented by nodes 202 and 204, when aligned so as to end at the last position of “AACC.” For example, as illustrated in Table 2 below, the state data may indicate that there is one error when aligning “AACC” and “A”, there are two errors when aligning “AACC” and “AA,” one error when aligning “AACC” and “AAC,” and no errors when aligning “AACC” and “AACC.”

TABLE 2
State Data (2^{nd} column) indicates the number of errors
between various prefixes of “AACCGA” (i.e., “A”,
“AA”, “AAC,” and “AACC”) and the sequence
“AACC” represented by nodes 202 and 204 of graph
reference construct 200 of FIG. 2, when the prefix is aligned to
the graph reference 200 so as to end at the last position of “AACC.”

| Partial Alignment Ending at “C,” | |
|---|---|
| which is the Last Position of “AACC” | State Data (# Errors) |
| AACC | 1 |
| A | |
| AACC | 2 |
| AA | |
| AACC | 1 |
| AAC | |
| AACC | 0 |
| AACC | |

FIG. 2B further illustrates aligning a biological sequence to a graph reference using an augmented version of a simple linear alignment algorithm. The linear alignment algorithm aligns a given sequence against a reference sequence by determining, for each position p in the reference sequence and for any length l, how accurately a length-l prefix of the given sequence matches the reference sequence when aligned to the reference sequence so as to end at position p. The augmented version of the linear alignment algorithm involves generating state data for each particular position of each sequence represented by the graph reference to indicate the number of errors k between a length-l prefix of the biological sequence and the graph reference, when the length-l prefix is aligned to graph reference so as to end at the particular position. The state data generated for a particular position may be used to generate state data for a subsequent position (either at the same or a subsequent node in the graph).

When two paths through the graph reference merge at a particular node (e.g., node 208), the state data from the two nodes preceding the particular node (e.g., nodes 204 and 206) to generate state data for a first position of the sequence represented by the particular node involves: (1) accessing state data for the nucleotide at the last position of the sequence represented by the first node (e.g., node 204) preceding the particular node (e.g., node 208), which may be termed “first state data”; (2) accessing state data for the nucleotide at the last position of the sequence represented by the second node (e.g., node 206) preceding the particular node (e.g., node 208), which may be termed “second state data”; and (3) generating state data for the first position of the sequence represented by the particular node (e.g., node 208) using the first state data and the second state data. The third step of generating the state data for the first position of the sequence represented by the particular node (e.g., node 208) may include merging the first state data and the second state data to obtain merged state data. In this illustrative example, merging state data involves, selecting for each prefix length l, the best partial alignment from among the incoming branches.

FIG. 2B shows illustrative examples of state data generated when aligning the target sequence “AACCGA” to graph reference construct 200 of FIG. 2A. For example, the state data 252 indicates, for each target sequence prefix with a length between 0 and 3, the number of errors between the prefix and the graph reference 200, when the prefix is aligned to end at the nucleotide “C” located at the last position of the sequence “AAC” represented by node 202. State data 254 indicates, for each target sequence prefix with a length between 0 and 4, the number of errors between the prefix and the graph reference 200, when the prefix is aligned to end at the nucleotide “C” represented by node 204. State data 254 may be generated at least in part by using state data 252, the sequence represented by node 204, and the target sequence. State data 256 indicates, for each target sequence prefix with a length between 0 and 5, the number of errors between the prefix and the graph reference 200, when the prefix is aligned to end at the nucleotide “C” at the last position of the sequence represented by node 206. State data 256 may be generated at least in part by using state data 252, the sequence represented by node 206, and the target sequence.

The state data for the first position of the sequence represented by node 208 is obtained in two steps: (1) a merging step during which the state data for the last positions of the sequences represented by nodes 204 and 206 (i.e., state data 254 and state data 256) is merged to obtained merged state data 258; and (2) an update step where the merged state (which does not depend on any of the nucleotides represented by node 208) is updated to generate state data 260, which takes the sequence represented by node 208 into account.

In some embodiments, after state data is generated for each of the positions of each sequence represented by each node in the graph reference, the generated state data may be used to obtain the best alignment (e.g., by tracking back calculations already performed as is typically done in dynamic programming).

It should be appreciated that although the augmented alignment technique described with above with reference to FIG. 2B generates gapless alignments, this augmented alignment technique may be generalized to produce alignments with gaps. For example, the state data may be augmented to store a shift distance indicating how many characters may be ignored on a given partial alignment. At a merging step, when merging different state data, the state data having the lowest shift distance may be selected. In this way, various linear alignment algorithms may be adapted to efficiently align sequences against graph references.

The inventors have recognized that another class of linear alignment algorithms that can be adapted to efficiently aligning sequences against graph references is the class of bit parallel automaton (BPA) alignment algorithms. BPA algorithms are fast linear alignment algorithms that allow not only for substitutions, but also for insertions, and deletions. The main idea behind BPA algorithms is to pack together character comparisons as bits in an integer. In light of recurrences between the bits, shifting and matching sequence patterns against one another may be performed using a small number of bitwise operations, which may be performed very quickly using computer processors since the bitwise operations are often implemented using native instructions on the computer processors. Aspects of conventional BPA algorithms for linear alignment are discussed in Sun Wu and Udi Manber, “Fast Text Searching with Errors,” University of Arizona, Department of Computer Science, TR 91-11, 1991, which is incorporated by reference herein in its entirety.

In some embodiments, an exact-matching implementation of a linear BPA aligner may operate as follows. Consider a pattern P={p₁, p₂, . . . , pₘ} and a text T={t₁, t₂, . . . , tₘ]. Let R be a bit array of size m. Rⱼ refers to the value of the array R after the j^{th} character in T has been processed. The array Rⱼ contains information about all matches of prefixes of P that end at j. In particular, Rⱼ[i]=1 if and only if the first i characters of P match exactly the last i characters up to j in the text T. When we read tⱼ₊₁, we need to determine whether tⱼ₊₁ can extend any of the partial matches so far. The transition from Rⱼ to Rⱼ₊₁ can be summarized as follows:

Initially, R₀[i]=0 for all i, 1≤i≤m; R₀[i]=0 (to avoid having a special case for i=1). Rⱼ[0]=1 if tⱼ=p₁. The remaining values of R may be filled in as follows:

$${R}_{j} [ i ] = \{ \begin{matrix}1 & if {R}_{j} [ i - 1 ] = 1 and {p}_{i} = {t}_{j + 1} \\ 0 & otherwise\end{matrix} .$$

In addition, this transition may be implemented faster by creating a bit mask for each character in the alphabet used by the pattern and performing a right shift of Rⱼ. As a result, each transition calculation in the linear BPA alignment algorithm may be executed using two simple bitwise operations: a logical bitwise shift and a bitwise AND operation. Given the values of the arrays Rⱼ (for 1≤j≤m), an exact match between P and T may be identified, whenever Rⱼ[i]=1.

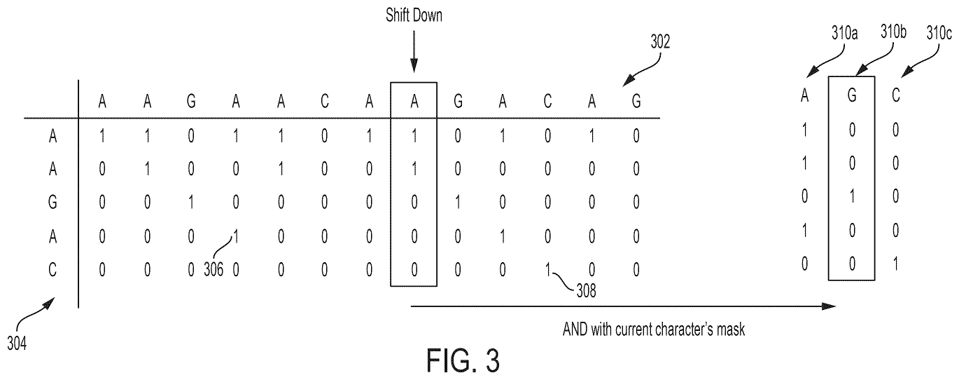

FIG. 3 is a diagram illustrating the application of a bit-parallel automaton (BPA) linear alignment technique to aligning the target sequence 304 consisting of the five nucleotides “AAGAC” to a reference sequence 302 consisting of the 13 nucleotides “AAGAACAAGACAG” (SEQ ID NO: 3). The columns of the 5×13 matrix shown in FIG. 3 are the bit arrays Rⱼ, (in this example with 1≤j≤13) which contains information about all matches of prefixes of the target sequence 304 and the reference sequence 302. For example, entry 306 of the matrix indicates that R₄[4]=1, which indicates that the first four nucleotides of target sequence 304 match the first four nucleotides of reference sequence 302. As another example, entry 308 of the matrix indicates that R₁₁[5]=1, which indicates that the target sequence 304 exactly matches the reference sequence 302, when the entire 5-nucleotide target sequence 304 is aligned to the reference sequence 302 so as to end at the 11^{th} position of the reference sequence.

This example further illustrates that in a linear BPA alignment technique, the bit array Rⱼ₊₁ can be obtained from the bit array Rⱼ using two bitwise operations: (1) first the array Rⱼ is shifted down (in some processors such shifting may be implemented using a native instructions such as a left- or a right-shift); and (2) a bitwise AND operation is computed between the shifted down array and a bit mask (e.g., one of the bit masks 310a, 310b, and 310c) corresponding to the nucleotide at position j+1 in the reference sequence 302. For example R₁₁ may be obtained from R₁₀ by shifting the bits of R₁₀ down and computing a bitwise AND between the shifted down bits and the bit mask 310c, which is associated with the nucleotide “C” at the position 11^{th} position in the reference sequence 302.

In some embodiments, the linear BPA alignment may be extended to allow approximate matching by allowing k substitutions, insertions, and deletions. This may be accomplished by storing k additional bit arrays R¹, R², and R^{k}, such that array Rd stores all possible matches with up to d errors. Determining the transition from array Rⱼ^{d} to Rⱼ^{d} involves evaluating the various cases of a match, substitution, insertion and deletion. Further details are described in Sun Wu and Udi Manber, “Fast Text Searching with Errors,” University of Arizona, Department of Computer Science, TR 91-11, 1991, which is incorporated by reference herein in its entirety.

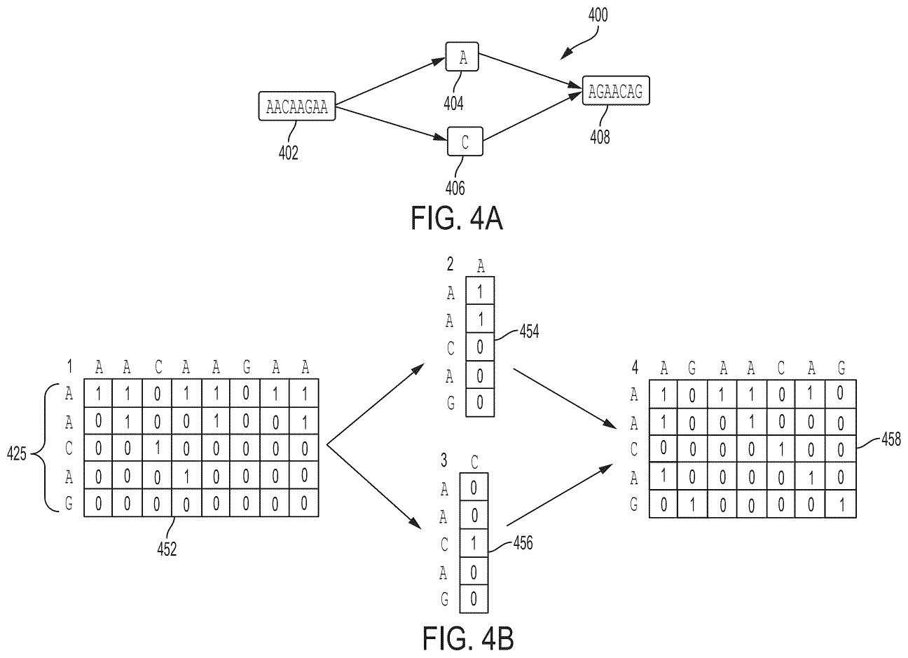

The inventors have recognized that the BPA linear alignment algorithm of Wu and Manber, or any known variation thereof may be adapted to efficiently to align biological sequences against graph references (i.e., without enumerating all the paths in a graph reference, which is intractable for many problems of interest for reasons discussed above). In some embodiments, an adapted BPA algorithm may be used to align a biological sequence against a graph reference by generating state data for each particular position of each sequence represented by the graph reference to indicate whether a length-l prefix of the biological sequence matches the graph reference exactly, when the length-l prefix is aligned to graph reference so as to end at the particular position. The adapted BPA algorithm is described in more detail below with reference to the graph reference construct 400 shown in FIG. 4A. Graph reference 400 includes node 402 (node “1”) representing the sequence “AACAAGAA”, node 404 (node “2”) representing the sequence “A”, node 406 (node “3”) representing the sequence “C”, and node 408 (node “4”) representing the sequence “AGAACAG”.

In some embodiments, the state data for a particular position p at node N may be represented by a bit array R_{p,N} and R_{p,N}[l]=1 when the length-l prefix of the biological sequence matches the graph reference exactly when aligned to the graph reference so as to end at the position p. FIG. 4B illustrates the state data generated when applying the adapted BPA algorithm to align the target sequence 425 “AACAG” to the graph reference 400. In particular, FIG. 4B shows matrix 452 which includes bit arrays for each nucleotide in the sequence represented by node 402, matrix 454 which includes a bit array for the nucleotide “A” represented by node 404, matrix 456 which includes a bit array for the nucleotide “C” represented by node 406, and matrix 458 which includes a bit array for each nucleotide in the sequence represented by node 408. The values of the array may be initialized to 0, as in the case of the linear BPA algorithm.

When computing the bit array R_{p,N} for a position p other than the first position in a sequence represented by a node N in the graph, the bit array R_{p,N} may be obtained by: (1) shifting down the bit array R_{p-1,N} representing the state data for the position p−1 in the sequence represented by the node N; and (2) computing a bitwise AND between the shifted down bit array and a bit mask associated with the nucleotide at the position p in the sequence represented by the node N. In addition, R_{p,N}[0]=1 if the first nucleotide of the target sequence is the same as the nucleotide at the pth position in the sequence represented by node N. For example, as shown in FIG. 4B, the fourth column of matrix 452, representing R_{4,1} may be obtained by: (1) setting R_{4,1}[0]=1 because the first nucleotide of target sequence 425 matches the nucleotide at the fourth position; (2) generating values for the remaining entries by computing a bitwise AND between a down shifted version of bit array R_{3,1} (the down shifted version is given by [00010]^{T}) and a bit mask for the nucleotide A (i.e., [1 1 0 1 0]^{T}).

When computing the bit array R_{1,N} for a first position in a sequence represented by a node N in the graph, which is immediately preceded by only single node M in the graph (i.e., the node N is not a merge point in the graph), the bit array R_{1,N} may be obtained by: (1) shifting down the bit array representing the state data for the last position of the node M; and (2) computing a bitwise AND between the shifted down bit array and a bit mask associated with the nucleotide at the first position in the sequence represented by the node N. In addition, R_{1,N}[0]=1 if the first nucleotide of the target sequence is the same as the nucleotide at the first position of the sequence represented by node N. For example, as shown in FIG. 4B, the column of matrix 454, representing R_{1,2} may be obtained by: (1) setting R_{1,2}[0]=1 because the first nucleotide of target sequence 425 matches the nucleotide at the ninth position (of the whole reference sequence going through node 404 from the beginning or, equivalently, the first nucleotide in the sequence represented by node 404); (2) generating values for the remaining entries by computing a bitwise AND between a down shifted version of bit array R_{1,N} (the downshifted version is given by [01100]^{T}) and a bit mask for the nucleotide A (i.e., [1 1 0 1 0]^{T}).

The last case to specify is how to handle a merging of two paths in the graph reference (e.g., how obtain the bit array R_{1,4}). When computing the bit array R_{1,N} for a first position in a sequence represented by a node N in the graph, which is immediately preceded by multiple other nodes, the bit array R_{1,N} may be obtained by: (1) computing merged state data from the bit arrays representing the state data for the last positions of each of the nodes preceding node N in the graph; (2) updating the merged state data to account for the nucleotide at the first position in the sequence represented by node N. The merged state data may be obtained by calculating a bitwise OR of the bit arrays representing the state data for the last positions of each of the nodes preceding node N in the graph. The merged state data may be updated by: (1) shifting the bit array of the merged state data; and (2) computing a bitwise AND between the shifted down bit array and a bit mask associated with the nucleotide at the first position in the sequence represented by the node N. In addition, R_{1,N}[0]=1 if the first nucleotide of the target sequence is the same as the nucleotide at the first position of the sequence represented by node N.

For example, as shown in FIG. 4B, the first column of matrix 458, representing R_{1,4} may be obtained by: (1) computing merged state data by calculating a bitwise OR of the bit arrays R_{1,2} (i.e., [1 1 0 0 0]^{T}) and R_{1,3} (i.e., [0 0 1 0 0]^{T}) to obtain merged state data (i.e., [1 1 10 0]^{T}); and (2) updating the merged state data to account for the nucleotide “A” at the first position of node 4 to obtain the bit array [1 1 0 1 0]^{T}. This second step involves: (1) setting R_{1,4}[0]=1 because the first nucleotide of target sequence 425 matches the first nucleotide in the sequence represented by node 404); (2) generating values for the remaining entries by computing a bitwise AND between a down shifted version of merged state data (the downshifted version is given by [01110]^{T}) and a bit mask for the nucleotide A (i.e., [1 1 0 1 0]^{T}) to obtain the array [1 1 0 1 0]^{T}.

In the above description of the augmented BPA alignment algorithm, the state data for a particular position p at node N may be R_{p,N}[l]=1 when the length-l prefix of the biological sequence matches the graph reference exactly when aligned to the graph reference so as to end at the position p. However, in other embodiments, the role of the “1” bit and the “0” bit may be reversed, so that, when the length-l prefix of the biological sequence matches the graph reference exactly when aligned to the graph reference so as to end at the position p, R_{p,N}[l]=0. In such embodiments, the bitwise operation “OR” may be used for generating state data instead of the bitwise operation “AND”. Similarly, during the merging step of calculating merged state data an “AND” operation may be used instead of the bitwise operation “OR.”

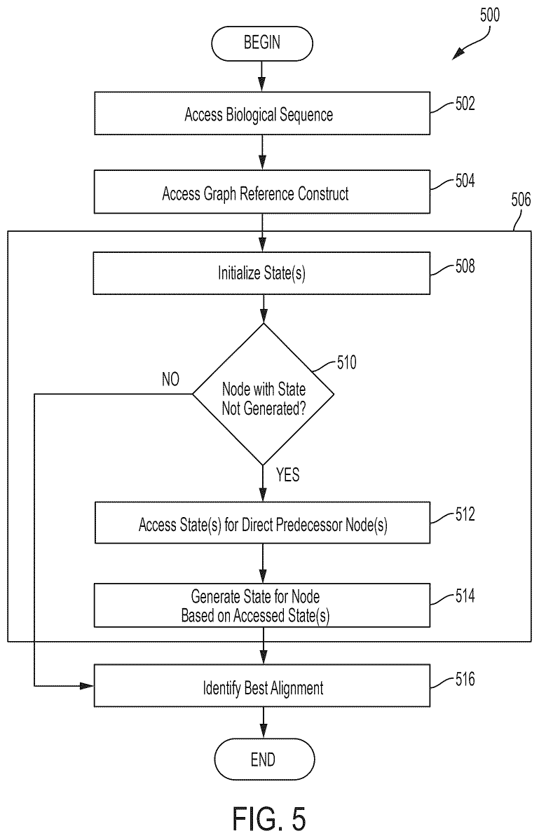

FIG. 5 is a flowchart of an illustrative process 500 for aligning a biological sequence to a graph reference construct, in accordance with some embodiments of the technology described herein. Process 500 may be performed by any suitable computing device(s) (e.g., a single computing device, multiple computing devices co-located in a single physical locations or located in multiple physical locations remote from one another, one or more computing devices part of a cloud computing system, etc.), as aspects of the technology described herein are not limited in this respect.

Process 500 begins at act 502, where a biological sequence is obtained. The biological sequence may be obtained by sequencing one or more biological samples obtained from an individual, for example, by using next generation sequencing and/or any other suitable sequencing technique or technology, as aspects of the technology described herein are not limited by the manner in which the biological samples for an individual are obtained.

Next, process 500 proceeds to act 504, where a graph reference construct is accessed. The graph reference construct may be embodied in a directed graph comprising a plurality of nodes and through which there are multiple paths. The directed graph may be embodied in one or more data structures of any suitable type, as aspects of the technology described herein are not limited in this respect. The graph reference may have been generated using any suitable graph reference construction technique including any of the techniques described in U.S. Patent Publication No. 2015-0057946, entitled “METHODS AND SYSTEMS FOR ALIGNING SEQUENCES,” published on Feb. 26, 2015, which is incorporated by reference herein in its entirety. In some cases, the directed graph may be a subset, or “local”, portion of a larger directed graph that has been identified as a likely region for alignment by a separate searching algorithm (e.g., a global search algorithm).

Next, process 500 proceeds to act 506, during which state data for each node in the graph reference is generated based, at least in part, on the sequence accessed at act 502. In some embodiments, state data may be generated for at least some (e.g., all) positions of each sequence represented by each node in the graph. The state data may be of any suitable type including any of the types described herein including with reference to FIGS. 2A-B, 3, and 4A-B. For example, state data for a nucleotide at a particular position in a sequence represented by a node in a graph reference may indicate an extent to which each of one or multiple subsequences (e.g., prefixes) of the biological sequence matches the graph reference construct when aligned to the graph reference construct so as to end at the particular position. An indication about the extent of a match between two sequence, for example, may indicate whether there is an exact match between the two sequences or how many errors there are between the two sequences when in a given alignment relative to one another.

In the illustrative process 500, state data may be generated iteratively in accordance with the structure of the graph reference, as described next with reference to act 508, decision block 510, act 512, and act 514. At act 508, the data structure(s) for storing generated state data may be initialized. This may be done in any suitable way. For example, in embodiments where the state data is stored using one or more bit arrays (e.g., arrays R_{p,N} described with reference to FIG. 3), the bit array(s) may be initialized (e.g., to the value 0).

Next, at decision 510, it may be determined whether there is any node for which state data has not been generated. In some embodiments, determining whether there is a node for which the state data has not been generated may comprise determining whether there is any node representing a nucleotide sequence where state data has not been generated for each and every nucleotide in the sequence. In some embodiments, determining whether there is any node for which state data has not been generated comprises iterating through the nodes in the graph (e.g., using breadth first search) to identify any nodes for which state data has not been generated. Thus, in some embodiments, state data may be generated iteratively for the nodes in the graph in accordance with the structure of the graph as iterated over in accordance with a breadth-first search or any other suitable technique for iterating over a structure of a directed graph.

When it is determined, at decision block 510, that state data has been generated for each and every node in the graph (so that there are no nodes for which the state data has not been generated), process 500 proceeds, via the NO branch, to act 516. On the other hand, when it is determined that there is a node N in the graph for which the state data has not been generated, act 500 proceeds, via the YES branch, to acts 512 and 514, where state data is generated for the node N.

At act 512, the state data for the node(s) directly preceding node N in the graph is accessed. In embodiments where node N is preceded by a single node, the state data for the nucleotide in the last position of the sequence represented by the single node may be accessed. In embodiments where node N is preceded by multiple nodes (e.g., when two or more paths through the graph merge at node N), the state data for the nucleotides in the last positions of the sequences represented by the multiple nodes may be accessed.

Next, at act 514, the state data accessed for the node(s) directly preceding node N may be used to generate the state data for node N. In some embodiments, the state data accessed for the node(s) directly preceding node N may be used to generate the state data for the nucleotide in the first position of the sequence represented by node N. Subsequently, the state data generated for the nucleotide in the first position of the sequence represented by node N may be used to generate state data for the other positions of the sequence represented by node N. After the state data is generated for a node, the state data may be stored for subsequent use (e.g., to generate state data for one or more other nodes in the graph and/or to determine the alignment between the sequence obtained at act 502 and the graph reference accessed at act 504).

When the node N is directly preceded by only a single node, the state data for the first position in the sequence represented by node N may be generated using the state data for the last position in the sequence represented by the preceding node; and the sequence obtained at act 502. This may be done in any suitable way and, for example, may be done using any of the alignment algorithms described with reference to FIGS. 2A-B, 3 and 4A-B. As one specific example, in this instance, the first state data may be generated using the transition logic for the augmented BPA alignment algorithm described with reference to FIG. 4A-B.

When the node N is directly preceded by multiple nodes, the state data for the first position in the sequence represented by node N, may be generated in two steps. First, the state data for the last positions of the sequences represented by the preceding nodes may be used to generate merged state data (e.g., as described herein with reference to 2A-B and 4A-B), and the merged state data may be used together with the sequence obtained at act 502 to generate the state data for the first position of the sequence represented by node N (e.g., as described herein with reference to FIGS. 2A-B and 4A-B).

After the state data is generated for each node in the graph, process 500 proceeds to act 516, where the best alignment between the sequence accessed at act 502 and the graph reference construct accessed at act 504 is identified. In some embodiments, the best alignment may be identified using the state data generated during act 506. For example, in some embodiments, the best alignment may be identified by tracking back calculations performed to generate the state data as is done in dynamic programming techniques. In some embodiments, this can include following pointers that indicate which portions of the state data are associated with previous nodes. After the alignment between the biological sequence and the graph reference construct is generated, process 500 completes.

When aligning a sequence to a reference construct, alignment errors may occur near one or both ends of the sequence. To reduce the impact of such errors, a so-called “soft clipping” technique, which allows for the discounting of errors that occur at one or both ends of the sequence when selecting an alignment. The mismatched portions may be “clipped” (omitted) from the final alignment and ignored when performing variant calling and/or any other sequence processing steps.

For example, as shown below, the mismatching bases (emphasized using underlining) at the end of the short read may be clipped from the alignment.

Reference sequence
(SEQ ID NO: 4)
GCTGATGTGCCGCCTCACTTCGGTGGTGAGGTG
Short read
(SEQ ID NO: 5)
CTGATGTGCCGCCTCACTTCGGTGGTACCGTA

The inventors have recognized that the BPA linear alignment algorithm, as well as the augmented BPA techniques described herein, may be extended to incorporate soft clipping. The inventors have appreciated that, in some embodiments, soft clipping may be integrated into the BPA linear algorithm by defining a function that calculates a soft clipping cost less than the cost of allowing an equivalent number of mismatches. For example, such a function may take the form:

$$soft\_cost ( M ) < M * mismatch\_cost ,$$

where the soft clipping cost function “soft_cost” generates a soft clipping cost for a portion of a sequence having length of M nucleotides, is smaller than the cost of M mismatches. The inventors have also recognized that in order to incorporate soft clipping into the BPA linear alignment algorithm, the left end of a sequence read needs to be treated differently from the right end of the sequence read. Accordingly, in some embodiments, a BPA linear alignment algorithm may be modified to by incorporating a soft-clipping technique for the right end of a sequence read and another soft-clipping technique for the left end of the sequence read, as discussed in more detail below.
Soft-Clipping from the Right

As discussed herein, in a linear BPA alignment algorithm, identifying an aligned position of a sequence read may be performed by finding a lowest mismatch d-bit array Rd having a “1” set in its final position. Accordingly, the aligned position of a sequence read according to the linear BPA algorithm may be found by solving:

$$arg {min}_{d \leq m ax \_ errors} ( {R}_{j}^{d} ⌈ read\_length ⌉ = 1 ) ,$$

where “read_length” represents the number of nucleotides in the sequence reads and “max_errors” specifies the maximum number of errors allowed. The inventors have recognized that soft-clipping a sequence read from the right is equivalent to ignoring one or more trailing “0” bits in a given bit array Rⱼ^{d}. Thus, finding a soft-clipped alignment from the right may be performed by finding the lowest mismatch bit array having a “1” set in a non-final position as follows:

$$arg {min}_{d \leq ma x \_ errors} ( soft\_cost ( read\_length - last\_set \_bit ( {R}_{j}^{d} ) ) + d ) .$$

Selecting an appropriate Rⱼ^{d} and position i may be performed by using the last_set_bit( ) function, which finds the position of the last “1” bit. The value calculated for “read_length-last_set_bit( )” corresponds to the number of bases to clip at the right end of the sequence read. The new total cost for that Rⱼ^{d} is the corresponding error count d, plus the added cost of the soft clip. As alignment proceeds, we keep track of the least total cost for each Rⱼ^{d}, and then return the error count and Rⱼ^{d} corresponding to the minimum.

For example, consider a 50 base pair read, and a corresponding Rⱼ⁵ (allowing d=5 mismatches) in which the last bit set to “1” is at the 40th base. In this example, the cost for soft clipping this alignment after the 40^{th} base is soft_cost (10)+5. This cost is stored for that Rⱼ⁵. These costs are tracked, and ultimately the Rⱼ^{d} having the lowest total cost is selected as a right soft-clipped aligned position.

Soft-Clipping from the Left

The inventors have recognized that soft-clipping from the left of a sequence read using a BPA linear alignment algorithm cannot work in the same way as the above-described technique for soft clipping on the right because each of the positions in each Rⱼ^{d} depends on Rⱼ₋₁^{d}. Instead, the solution is to set the left-most bits to “1” when the error count d and soft clip costs are equivalent. These positions can be identified as follows. Consider the set SL of pairs (i, d) corresponding to all bit arrays Rⱼ^{d}[i], for which there are two options: either there exists an actual alignment of the prefixes within d errors (meaning the bits are already set to 1), or we can soft clip because the soft clip cost is at most d. In the latter case, all of these bits are then set to 1 by default, allowing subsequent calculations of Rⱼ₊₁^{d}[i+1] to incorporate these bits to continue the alignment. The set SL may be defined as follows:

$$SL = \{ ( i , d ) \vert i \leq read\_length , d \leq max\_errors , soft\_cost ( i ) \leq d \} .$$

For example, consider a possible aligned position for a 50 bp read that begins with a stretch of 5 bases (emphasized by underlining) having 3 mismatches:

Reference sequence
(SEQ ID NO: 4)
GCTGATGTGCCGCCTCACTTCGGTGGTGAGGTG
Short read
(SEQ ID NO: 6)
ACGAGGTGCCGCCTCACTTCGGTGGTGAGGTG

Let each position j in the reference be represented by a bit array Rⱼ⁵. Positions within each Rⱼ⁵ may then be evaluated to determine whether any positions j within the array meets the criteria specified above. If so, these bits are set to “1”, allowing for an extension of the alignment from that position. Once the costs for soft-clipping left according to a given function are calculated, they may be applied either as a set of static masks for each d, or incorporated as a template Rⱼ^{d}.

It should be appreciated that the above-described “left” and “right” soft clipping techniques are not limited to being incorporated in linear BPA alignment algorithms and, in some embodiments, may be used to extend the augmented BPA alignment algorithm described herein (for aligning sequences against graph reference constructs) to perform soft clipping.

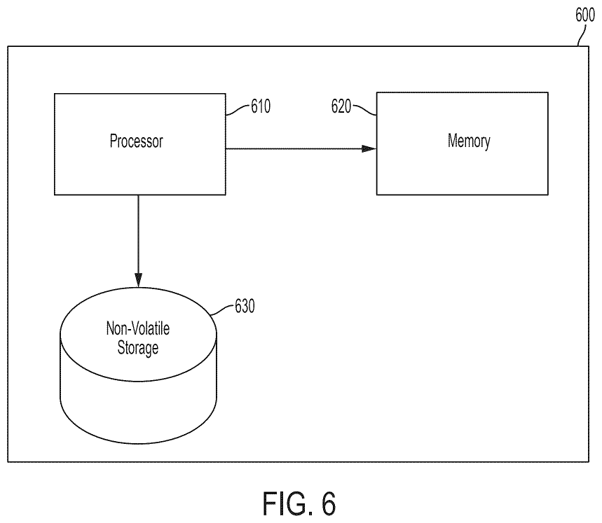

An illustrative implementation of a computer system 600 that may be used in connection with any of the embodiments of the disclosure provided herein is shown in FIG. 6. The computer system 600 may include one or more processors 610 and one or more articles of manufacture that comprise non-transitory computer-readable storage media (e.g., memory 620 and one or more non-volatile storage media 630). The processor 610 may control writing data to and reading data from the memory 620 and the non-volatile storage device 630 in any suitable manner, as the aspects of the disclosure provided herein are not limited in this respect. To perform any of the functionality described herein, the processor 610 may execute one or more processor-executable instructions stored in one or more non-transitory computer-readable storage media (e.g., the memory 620), which may serve as non-transitory computer-readable storage media storing processor-executable instructions for execution by the processor 610.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided including with reference to FIG. 5. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. A system, comprising:
at least one computer hardware processor; and
at least one non-transitory computer-readable storage medium storing processor-executable instructions that, when executed by the at least one computer hardware processor, cause the at least one computer hardware processor to perform:
accessing a biological sequence;
accessing a graph reference construct representing a graph through which there are multiple paths including a first path and a second path, the graph comprising a plurality of nodes including first, second, and third nodes, the first node preceding the third node along the first path, and the second node preceding the third node along the second path;
aligning the biological sequence to the graph reference construct, the aligning comprising:
accessing first state data indicating an extent to which each of multiple subsequences of the biological sequence matches the graph reference construct when aligned to the graph reference construct so as to end at a last position of a sequence represented by the first node;
accessing second state data indicating an extent to which each of the multiple subsequences of the biological sequence matches the graph reference construct when aligned to the graph reference construct so as to end at a last position of a sequence represented by the second node;
generating third state data using the first state data and the second state data, the third state data indicating an extent to which each of the multiple subsequences of the biological sequence matches the graph reference construct when aligned to the graph reference construct so as to end at a first position of a sequence represented by the third node; and
storing the third state data.

2. The system of claim 1, wherein the multiple subsequences of the biological sequence include a first subsequence, and wherein generating the third state data comprises:
determining a number of errors between the first subsequence and the graph reference construct, when the first subsequence is aligned to the graph reference construct so as to end at the first position of the sequence represented by the third node; and
including a value indicating the determined number of errors in the third state data.

3. The system of claim 2, wherein determining the number of errors comprises:
determining a first number of errors between the first subsequence and the graph reference construct, when the first subsequence is aligned to the graph reference construct so as to end at the last position of the sequence represented by first node;
determining a second number of errors between the first subsequence and the graph reference construct, when the first subsequence is aligned to the graph reference construct so as to end at the last position of the sequence represented by the second node; and
determining the number of errors based on a minimum of the first number of errors and the second number of errors.

4. The system of claim 1, wherein the multiple subsequences of the biological sequence include a first subsequence, and wherein generating the third state data comprises:
determining whether the first subsequence matches the graph reference construct exactly when aligned to the graph reference construct so as to end at the first position of the sequence represented by the third node; and
including a value indicating a result of the determination in the third state data.

5. The system of claim 2, wherein the value is a 0 or a 1.

6. The system of claim 1, wherein the first state data includes first binary data, the second state data includes second binary data, and the third state data includes third binary data, and wherein generating the third state data comprises:
generating the third binary data by applying at least one bitwise operation to the first binary data and the second binary data.

7. The system of claim 6, wherein the at least one bitwise operation comprises a bitwise OR operation.

8. The system of claim 6, wherein the at least one bitwise operation comprises a bitwise AND operation.

9. The system of claim 1, wherein generating the third state data comprises:
for each one of the multiple subsequences,
generating a respective binary value indicating whether the each one multiple subsequence exactly matches the graph reference construct when aligned to the graph reference construct so as to end at the first position of the sequence represented by third node; and
including the respective binary value in the third state data.

10. The system of claim 1, wherein the sequence represented by the third node consists of a single nucleotide, wherein the plurality of nodes includes a fourth node following the third node in the graph, and wherein the aligning further comprises:
accessing the third state data;
generating fourth state data using the third state data, the fourth state data indicating an extent to which each of the multiple subsequences of the biological sequence matches the graph reference construct when aligned to the graph reference construct so as to end at the fourth node; and
storing the fourth state data.

11. The system of claim 1, wherein the sequence represented by the third node consists of multiple nucleotides including, and wherein the aligning further comprises:
accessing the third state data;
generating fourth state data using the third state data, the fourth state data indicating an extent to which each of the multiple subsequences of the biological sequence matches the graph reference construct when aligned to the graph reference construct so as to end at a second position of the sequence represented by the third node; and
storing the fourth state data.

12. The system of claim 1, wherein the aligning further comprises:
for each position of each subsequence represented by a respective node in the plurality of nodes,
generating respective state data indicating an extent to which each of the multiple subsequences of the biological sequence matches the graph reference construct when aligned to the graph reference construct so as to end at the position.

13. A method, comprising:
using at least one computer hardware processor to perform:
accessing a biological sequence;
accessing a graph reference construct representing a graph through which there are multiple paths including a first path and a second path, the graph comprising a plurality of nodes including first, second, and third nodes, the first node preceding the third node along the first path, and the second node preceding the third node along the second path;
aligning the biological sequence to the graph reference construct, the aligning comprising:
accessing first state data indicating an extent to which each of multiple subsequences of the biological sequence matches the graph reference construct when aligned to the graph reference construct so as to end at a last position of a sequence represented by the first node;
accessing second state data indicating an extent to which each of the multiple subsequences of the biological sequence matches the graph reference construct when aligned to the graph reference construct so as to end at a last position of a sequence represented by the second node;
generating third state data using the first state data and the second state data, the third state data indicating an extent to which each of the multiple subsequences of the biological sequence matches the graph reference construct when aligned to the graph reference construct so as to end at a first position of a sequence represented by the third node; and
storing the third state data.

14. The method of claim 13, wherein the multiple subsequences of the biological sequence include a first subsequence, and wherein generating the third state data comprises:
determining whether the first subsequence matches the graph reference construct exactly when aligned to the graph reference construct so as to end at the first position of the sequence represented by the third node; and
including a value indicating a result of the determination in the third state data,
wherein the value is a 0 or a 1.

15. The method of claim 13, wherein the first state data includes first binary data, the second state data includes second binary data, and the third state data includes third binary data, and wherein generating the third state data comprises:
generating the third binary data by applying at least one bitwise operation to the first binary data and the second binary data.

16. The method of claim 15, wherein the at least one bitwise operation comprises a bitwise OR operation.

17. The method of claim 15, wherein the at least one bitwise operation comprises a bitwise AND operation.

18. At least one non-transitory computer-readable storage medium storing processor-executable instructions that, when executed by at least one computer hardware processor, cause the at least one computer hardware processor to perform:
accessing a biological sequence;
accessing a graph reference construct representing a graph through which there are multiple paths including a first path and a second path, the graph comprising a plurality of nodes including first, second, and third nodes, the first node preceding the third node along the first path, and the second node preceding the third node along the second path;
aligning the biological sequence to the graph reference construct, the aligning comprising:
accessing first state data indicating an extent to which each of multiple subsequences of the biological sequence matches the graph reference construct when aligned to the graph reference construct so as to end at the first node;
accessing second state data indicating an extent to which each of the multiple subsequences of the biological sequence matches the graph reference construct when aligned to the graph reference construct so as to end at the second node;
generating third state data using the first state data and the second state data, the third state data indicating an extent to which each of the multiple subsequences of the biological sequence matches the graph reference construct when aligned to the graph reference construct so as to end at the third node; and
storing the third state data.

19. The at least one non-transitory computer-readable storage medium of claim 18, wherein the multiple subsequences of the biological sequence include a first subsequence, and wherein generating the third state data comprises:
determining whether the first subsequence matches the graph reference construct exactly when aligned to the graph reference construct so as to end at the first position of the sequence represented by the third node; and
including a value indicating a result of the determination in the third state data,
wherein the value is a 0 or a 1.

20. The at least one non-transitory computer-readable storage medium of claim 18, wherein the first state data includes first binary data, the second state data includes second binary data, and the third state data includes third binary data, and wherein generating the third state data comprises:
generating the third binary data by applying at least one bitwise operation to the first binary data and the second binary data.
